(19) United States
(12) Patent Application Publication
(10) Pub. No.: US 2014/0358587 A1
(43) Pub. Date: Dec. 4, 2014

(54) EMERGENCY RESPONSE PROTOCOL RECOMMENDER

(71) Applicant: KONINKLIJKE PHILIPS N.V., EINDHOVEN (NL)

(72) Inventors: Hanqing Cao, Mahwah, NJ (US); Vikrant Suhas Vaze, White Plains, NY (US); Saeed Bagheri, Ctoton on Hudson, NY (US)

(73) Assignee: KONINKLIJKE PHILIPS N.V., EINDHOVEN (NL)

(21) Appl. No.: 14/294,186

(22) Filed: Jun. 3, 2014

Related U.S. Application Data
(60) Provisional application No. 61/830,668, filed on Jun. 4, 2013.

(51) Int. Cl. A61B 5/00; G06F 19/00

(52) U.S. Cl. CPC A61B 5/0002; G06F 19/322
USPC 705/3

(57) ABSTRACT

A method for recommending customized emergency response for a subject includes receiving an indication that the subject requests emergency response, determining a customized emergency response protocol for the subject, based at least on a current physiological state of the subject, wherein the protocol is different from subject to subject, equipping an emergency response vehicle based on the customized emergency response protocol, and dispatching the emergency response vehicle to the subject.

The following generally relates to emergency response and more particularly determining customized emergency response protocol for a subject and recommending the customized emergency response protocol.

The importance of a rapid and efficient medical emergency response to home healthcare patients cannot be underestimated as it might be the difference between life and death. A common tenet is that faster response leads to better patient outcomes. Currently, the response to an emergency medical problem is general and generic to a subject, and sometimes the ambulance may be delayed by traffic or severe weather. Consequently, the medical care delivered to the subject may be delayed or even not available when the subject arrives at the emergency department. This may result in unnecessarily worse patient outcome and unnecessarily extra healthcare cost.

Aspects described herein address the above-referenced problems and others.

The following describes an approach for recommending personal emergency response for a subject. The approach utilizes a current physiological state of a subject and at least one of a past history of the subject or one or more past histories of one or more other subjects to determine a set of unique risk factors and risk scores that are matched with other similar subjects, with the result facilitating proactive action item emergency response recommendation, which can be used to customize the emergency response to the subject.

In one aspect, a method for recommending customized emergency response for a subject includes receiving an indication that the subject requests emergency response, determining a customized emergency response protocol for the subject, based at least on a current physiological state of the subject, wherein the protocol is different from subject to subject, equipping an emergency response vehicle based on the customized emergency response protocol, and dispatching the emergency response vehicle to the subject.

In another aspect, an emergency response system includes a data retriever including a current subject current state retriever that receives an indication that a subject requests emergency response, and an emergency response recommender that determines a customized emergency response protocol for the subject, based on a current physiological state of the subject, wherein the protocol is different from subject to subject, wherein an emergency response vehicle is equipped based on the protocol and dispatched to the subject.

In another aspect, a computer readable storage medium is encoded with computer readable instructions. The computer readable instructions, when executed by a processer, causes the processor to: obtain a current state of a subject, historical data about the subject, and historical data about one or more other subjects, extract risk factors for the subject and the one or more other subjects based on the current physiological state, the historical data about the subject, and the historical data about the one or more other subjects, determine risk scores for the subject and the one or more other subjects based on the risk factors for the subject and the one or more other subjects, determine a similarity measure between the risk score of the subject and risk scores of each of the one or more other subjects, rank the similarity measures between the risk scores from one of most similar to least similar or least similar to most similar, retrieve diagnoses for a sub-set of the one or more other subjects corresponding a predetermined number of the highest ranked similarity measures, identify a candidate diagnosis of the diagnoses by determining a product of a prevalence of the diagnoses and a similarity of the subject to the one or more the subjects and identifying the product with a maximum value, wherein the candidate diagnosis is the candidate diagnosis corresponding to the identified maximum value, generate a customized emergency response protocol for the subject based on the candidate diagnosis.

The invention may take form in various components and arrangements of components, and in various steps and arrangements of steps. The drawings are only for purposes of illustrating the preferred embodiments and are not to be construed as limiting the invention.

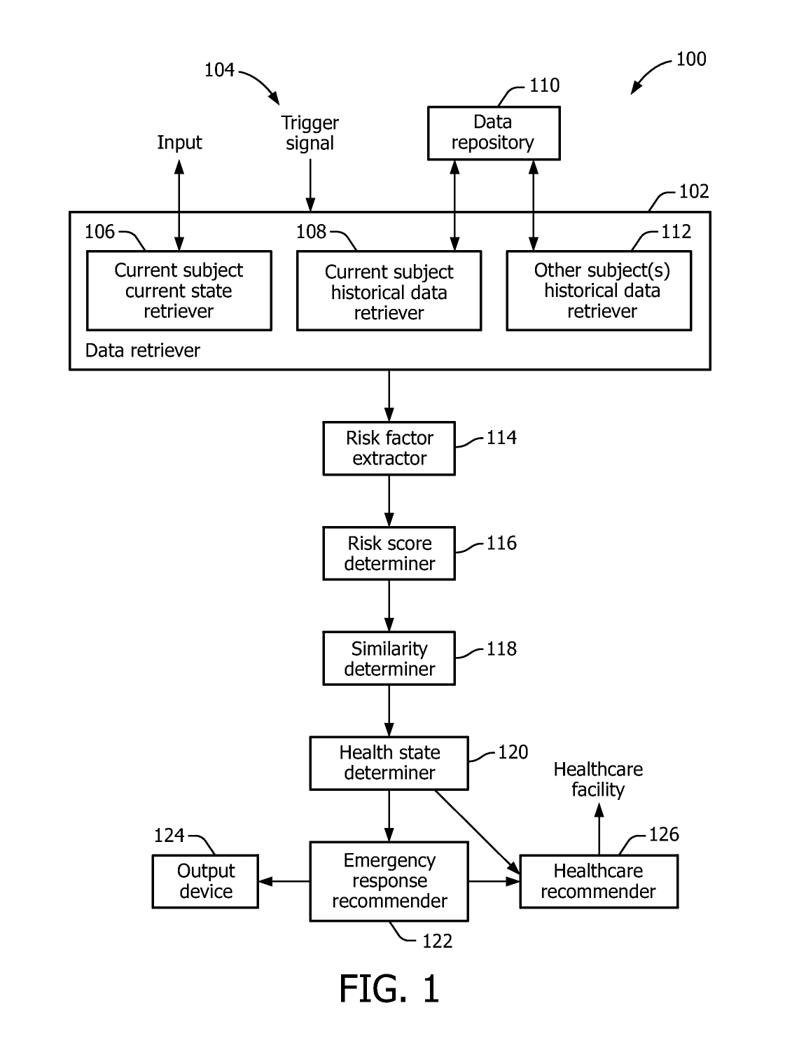

FIG. 1 schematically illustrates an example emergency response system.

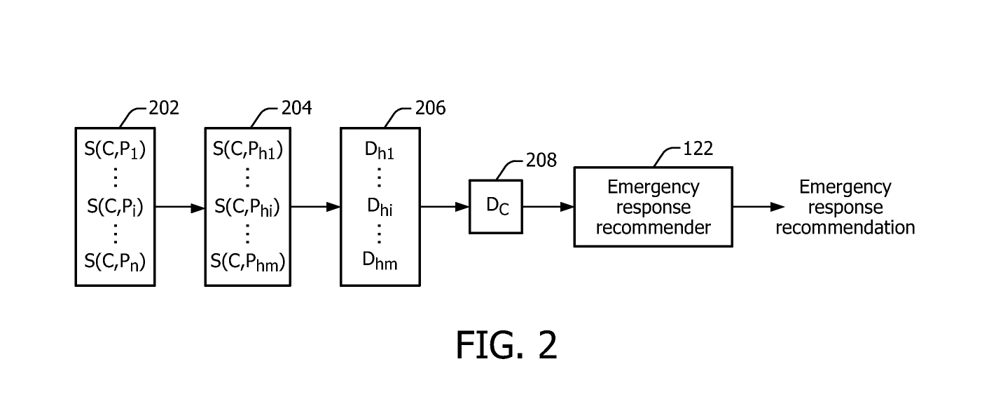

FIG. 2 illustrates an example method for recommending a customized emergency response protocol.

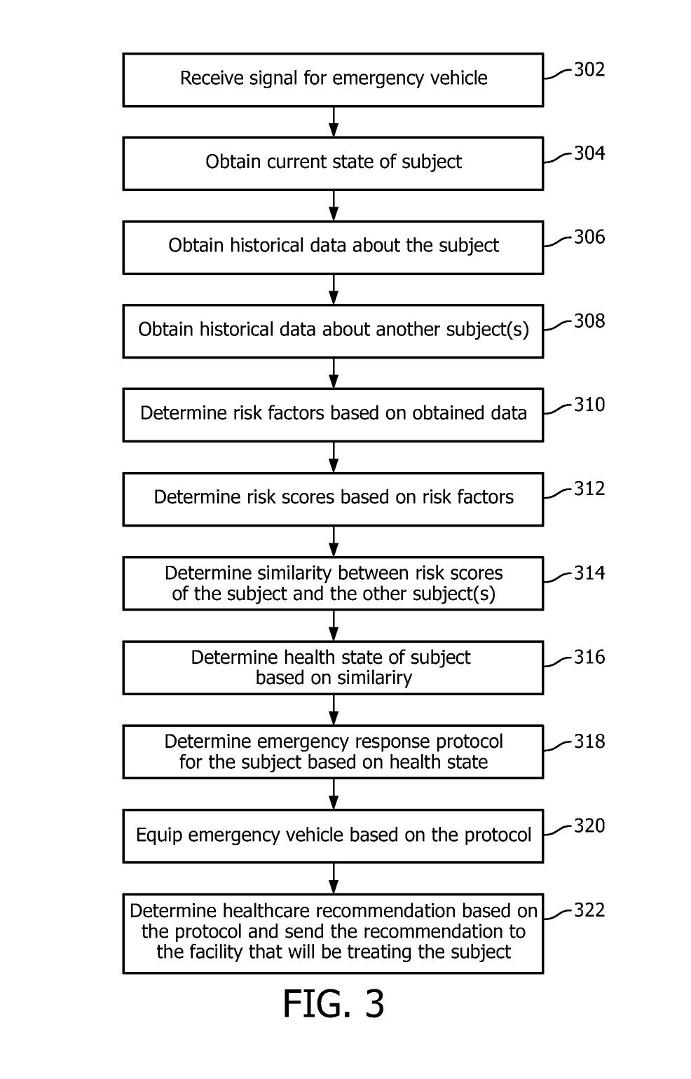

FIG. 3 illustrates the method of FIG. 2 in operation.

FIG. 1 illustrates an example emergency response system 100.

It is to be appreciated that the system 100 can be implemented with one or more computer processors (e.g., a microprocessor(s) and/or the like) executing one or more computer readable instructions stored on computer readable medium such as physical memory or other non-transitory medium. Additionally or alternatively, at least one of the instructions can be carried by a signal, carrier wave or other transitory medium.

The system 100 includes a data retriever 102. The data retriever 102 receives, as an input, a trigger signal 104, which invokes the data retriever 102 to retrieve data. In the illustrated example, the trigger signal is an emergency response notification such as a 9-1-1 call for a subject, a call from a manually and/or automatically activated alert device (e.g., Lifeline®, a trademark of Royal Philips Electronics of the Netherlands), etc.

In response to receiving the trigger signal 104, a current subject current state retriever 106 retrieves information about a current state of the subject (input). A source of the information can be the subject him/herself, a person/people present with the subject, a person/people remote from the subject (e.g., a clinician(s)), a combination of the foregoing, and/or other source.

Such information can be obtained in connection with the trigger signal 104. For example, where the trigger signal 104 is a telephone call, the current subject current state retriever 106 can obtain (and optionally record) the telephone call and utilize transcription and/or other software to transcribe audio of the telephone call and generate an alpha-numeric and/or textual version of the telephone call in an electronic format.

Examples of the information include, but are not limited to, an identification of the subject (e.g., a name, a social security number, etc.), a physiological symptom (e.g., a non-normal feeling observed by the subject), a physiological sign (e.g., an observable medical characteristic of the subject), a vital sign (e.g., temperature, blood pressure, heart rate, pulse, respiratory rate, etc.), a demographic (e.g., age, gender, ethnicity, etc.), a medical condition (e.g., disease), social-economic data, geographic data, and/or other information.

A current subject historical data retriever 108 retrieves historical data of the subject. In the illustrated example, this information is retrieved from a data repository 110. Such data may include an electronic medical record (EMR), a home medical record, and/or other record of historical data of the subject and/or one or more other subjects. The retrieved data may include, but is not limited to, medical data, chronic conditions, family history, and/or other data.

An other subject(s) historical data retriever 112 retrieves historical data of one or more other subjects. In the illustrated example, this information is retrieved from a data repository 110. In another example, this information is retrieved from a different data repository. Likewise, such data may include an electronic medical record (EMR), a home medical record, and/or other record of historical data of the subject and/or one or more other subjects. Such data may include, but is not limited to, medical data, chronic conditions, family history, etc.

A risk factor extractor 114 extracts one or more risk factors from the information and/or data obtained by the data retriever 102. Generally, a risk factor, as used herein, refers to any data item, original and/or derived, that is relevant to a subject's diagnosis and/or condition, and/or useful in determining action items. By way of non-limiting example, a risk factor can include demographic data, family history, past medical history, social-economic conditions, geographic factors, etc.

A risk factor can also include a relevant variable(s) that is derived from the information and/or data obtained by the data retriever 102. For example, a derived risk factor can include, but is not limited to, an integration or combination of both a risk(s) from past medical history and a risk(s) from current conditions/triggers. The following describes non-limiting examples of determining risk factors for a subject.

In one non-limiting example, a list of risk factors from past medical history of a potential heart attack include: age >=45, smoking, diabetes, hypertension, high blood cholesterol/triglyceride levels, family history of heart attack, obesity, lack of physical activity, stress/surgery, and etc., and a list of risk factors from current condition of a subject include stress and anger; in both cases, stress hormones are released to constrict blood vessel and blood flow and cause heart attack.

In another non-limiting example, a potential derived variable could be “post-surgical heart attack”, if the current condition is heart attack, and he had a surgery a week ago (past history). This derived variable connects a current condition (i.e., heart attack) with his/her recent past medical history (i.e., surgery), and is a different medical condition than heart attack without prior surgery. The treatment and preparation for treatment of this type of heart attack may be different from other types of heart attacks.

A risk score determiner 116 determines one or more risk scores based on the extracted risk factors. A risk score can be assigned to each subject with regard to a medical condition as a “composite risk factor.” For example, where subject A has 4 of the risks of heart attack (e.g., smoking, diabetes, hypertension, high blood cholesterol/triglyceride levels), and patient B has only 2 of the risks of heart attack (e.g., family history of heart attack, smoking), a risk score can be determined as shown below:
R_{A}=4 and R_{B}=2, respectively for patients A and B, respectively to represent their risk scores.
A composite risk score is determined as a weighted sum of the risk factors by odds ratio:

R_{A}=w_smoking*(smoking=yes)+w_diabetes*(diabetes=yes)+w _hypertension*(hypertension=yes)+w_lipids(lipids=high)

and

R_{B}=w_{—}famAMI*(famAMI=yes)+w_smoking*(smoking=yes)
where w_smoking, w_diabetes, w_hypertension, w_lipids, w_famAMI are weights of each individual risk factor of heart attack, and R_{A} and R_{B} are composite risk scores for patients A and B, respectively. These weights can be calculated from the odds ratios of each risk factor.
A composite risk score is determined as a non-linear combination of the risk factors by taking into account the correlation structure of all the risk factors. For example, hypertension is correlated with high blood lipids and diabetes, and the composite risk score for patient A could be:

R_{A}=w_smoking*(smoking=yes)+w_diabetes*(diabetes=yes)+w_hypertension*(hypertension=yes)+w_lipids(lipids=high)+w_diab_{—}HPN(diabetes=yes)*(hypertension=yes)+w_lipids_{—}HPN(hypertension=yes)*(lipids=high)+w_diab_lipids_{—}HPN(diabetes=yes)*(lipids=high)*(hypertension=yes)
where w_diab_HPN, w_lipid_HPN, and w_diab_lipids_HPN are weights derived from the correlation structure of the risk factors.

A similarity determiner 118 compares information of the current subject with information of the other subject(s) and determines a similarity there between. For example, suppose there are r risk factors, denoted as Rₖ., and as explained herein, risk factors can include demographic data, family history, past medical history, social-economic conditions and even geographic factors.

A similarity measure between the current patient and the i^{th} patient Pᵢ can be determined as a weighted sum of each “per risk factor” similarity measure, and is shown in EQUATION 1:

$$S ( C , {P}_{i} ) = {\sum }_{k = 1}^{r} {w}_{k} {S}_{k} ( {R}_{c , k} , {R}_{i , k} ) , \qquad EQUATION 1$$

where Sₖ(R_{c,k}, R_{i,k}) is a similarity measure of each risk factor Rₖ between the current patient C with risk factor value of R_{c,k} and the i^{th} patient Pᵢ, with risk factor value of R_{i,k}, and wₖ is the weight assigned to risk factor Rₖ. FIG. 2, shows calculation and storage of the similarity measure between the current patient and n patients as a vector, list or the like at 202.

The similarity measure Sₖ (R_{c,k}, R_{i,k}) of each risk factor Rₖ for two patients can be defined as the reciprocal of a function of the distance between the two risk factors, as shown in EQUATION 2:

$${S}_{k} ( {R}_{c , k} , {R}_{i , k} ) = \frac{1}{1 + dist ( {R}_{c , k} , {R}_{i , k} )} = \frac{1}{1 + \vert {R}_{c , k} - {R}_{i , k} \vert} . \qquad EQUATION 2$$

After calculating S(C,Pᵢ) for each medical condition of each of the other subject Pᵢ, the other subjects can be ranked based on similarity measures with the current subject. FIG. 2, shows a ranking of the other subject at 204.

Then, a predetermined number of patients (e.g., 3, <5, etc.) whose similarity with the current patient are among the highest can be identified. These patients are most similar to the current patient in risk factors, and their diagnosis and treatment could be used as a short-list of candidate diagnosis and treatment for the current patient. FIG. 2, shows an example short-list at 206.

A health state determiner 120 utilizes the short-list to determine a health state or “preliminary diagnosis” as a guide to narrow down diagnostic testing, aid final diagnosis, and expedite logistic, treatment and resource planning An example of this is where D_{c} is a preliminary candidate diagnosis for the current patient determined from Dₕ₁, . . . , Dₕᵢ, . . . , Dₕₘ, the diagnosis from similar patients Pₕ₁, . . . , Pₕᵢ . . . , Pₕₘ, where n is the number of all the other patients in the database, m is the number of patients selected with highest similarity to the current patient (e.g., 3, 5, 20, et.). FIG. 2, shows an example of D_{c} at 208.

In particular, one preliminary candidate diagnosis could be chosen based on prevalence and similarity. For example, the candidate diagnosis that when the product of the “prevalence of the diagnosis pₕᵢ” and “the similarity of the current patient C and the similar patient Pₕᵢ” is maximized, as shown in EQUATION 3:

$${D}_{c} = {max}_{i} [ {p}_{hi} * S ( C , {P}_{hi} ) ] , \qquad EQUATION 3$$

where Pₕᵢ refers to similar patients only (therefore the similarity is guaranteed). That is, two patients are similar in a specific medical condition if S(C,Pₕᵢ)<thd(med_cond), where thd is a pre-specified threshold of similarity measure for a certain medical condition (med_cond).

An emergency response recommender 122 processes the preliminary candidate diagnosis and recommends an emergency response protocol. The protocol is conveyed to an output device 124 such as a display monitor, a printer, memory, a computing system (e.g., a computer, a tablet computer, a smartphone, etc.). The output protocol is used to determine the devices and/or personnel equipped in an emergency response vehicle (e.g., an ambulance, a helicopter, etc.) and thus customize the emergency response to the subject.

Of course, the emergency response vehicle can be equipped otherwise. For example, authorized personnel can override the protocol, adding equipment and/or personnel not identified in the protocol and/or not including equipment and/or personnel identified in the protocol. The foregoing provides a unique and personal approach for emergency response to home healthcare and/or other subjects that may lead to cost reduction, improved time efficiency, improve subject outcomes, etc., relative to approaches not utilizing the approach described herein.

An optional healthcare recommender 126 processes the health state of the subject and/or the identified emergency response protocol and generates a recommendation, which is conveyed to a healthcare facility, such as the healthcare facility to which the emergency vehicle is taking the subject to and/or other healthcare facility. This can be achieved when the data of the current subject is sufficient for clinicians to take proactive actions, e.g., when the symptoms and subject conditions are straightforward.

In other more complex situations, data mining similar patients as a way to guide the current subject's diagnosis and treatment could be utilized. For instance, similar subjects' data can be searched, risk factors developed, for example, as described herein, and a recommended solution/action items to the problem can be determined by prioritizing the similarity (e.g., described above) of the “past other patients” and “the current patient”. The recommended solution and action items can be considered proactively at the emergency care.

By way of non-limiting example, when the healthcare facility receives subject data during and/or after a 9-1-1 call, the proactive actions my include: (1) decide the clinician's specialty, (2) identify the clinician available, (3) equip the ambulance with the medical device that is chosen for testing and preliminary diagnosis for this particular patient, (4) send the patient-customized ambulance to the patient. All these proactive action items are designed and aimed so that the subject can be taken care of sooner by the right clinician, using the right medical equipment.

Generally, the approach described herein utilizes a current subject's past and current clinical and non-clinical data, clinical and non-clinical records of other similar subjects, a set of unique risk factors and risk scores determined based thereon, a set of similarity models that match other similar subjects with the current subject, and a proactive action item recommendation based on the similarity measures. All these measures can be adjusted indirectly and intelligently through inputs from patient self-reporting, physicians and family members.

FIG. 3 illustrates an example method in accordance with the disclosure herein.

It is to be appreciated that the ordering of the acts is not limiting. As such, other orderings are contemplated herein. In addition, one or more acts may be omitted and/or one or more additional acts may be included.

At 302, a trigger signal indicating a subject may be in need of an emergency vehicle is received.

At 304, a current physiological state of the subject is obtained as described herein.

At 306, historical data about the subject is determined as obtained herein

At 308, historical data about one or more other subjects is obtained as described herein.

At 310, one or more risk factors are determined based on the obtained data as described herein.

At 312, one or more risk scores are determined based on the risk factors as described herein.

At 314, a similarity of the subject with the one or more other subjects is determined as described herein.

At 316, a health state of the subject is determined based on the similarity as described herein.

At 318, a protocol for equipping an emergency response vehicle for the subject is determined as described herein.

At 320, the emergency response vehicle is equipped based on the protocol and dispatched.

At 322, a healthcare recommendation is determined based on the protocol and is sent to the healthcare facility that will be treating the subject.

At least a portion of the above may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

The following provides a scenario without using the system 100.
8:00 A patient suffered from a sudden cardiac arrest at home. A family member performed CPR and his heart resumed beating.
8:05 The family member called 911.
8:15 An ambulance arrived
8:20 The patient arrived at the hospital
8:25 His ECG was collected and diagnosed
8:35 It was decided that he had to be sent to the cath lab.
8:45 The cath lab became available.
9:00 Coronary angiogram was performed;
9:10 Diagnosis was made based on angiogram and a treatment plan was made;
9:20 Started to prepare for CABG surgery.
9:50 Preparation for the surgery done; started surgery.
In this scenario, the patient survived the surgery, and some portion of his cardiac muscle has been permanently damaged between the time of the collapse and the surgery (1 hour 50 minutes, or 110 minutes), and after surgery, he stayed in the hospital for 7 days before being sent back to home.

The following provides a scenario with using the system 100.
8:00 A patient suffered from a sudden cardiac arrest at home. A family member performed CPR and his heart resumed beating.
8:05 The family member called 911.
8:10 The patient historical medical data and the current condition data were sent to the hospital; Given the patient was 70 years old, had a history of hypertension and peripheral artery disease, our tool found the patients matched most with these risk factors needed a CABG. Therefore, there was a large chance that the patient would need a catheterization testing and a surgery of CABG. Preparation for angiogram and CABG started.
8:20 A customized ambulance equipped with a cardiologist and ECG machine arrived;
8:25 ECG data collected in the ambulance; Cath lab was made ready in the hospital; The patient arrived at the hospital
8:40 Coronary angiogram was done; CABG surgery preparation was done
8:50 Preliminary diagnosis was confirmed; started surgery
In this scenario, the patient survived the surgery and no damage to the heart had been caused by this short delay between collapse and surgery (50 minutes); after surgery he stayed in the hospital for 4 days before being sent back to home.

In the above scenario, compared to the scenario without using our tool, a good hour has been saved because of the early preliminary diagnosis using our tool by extracting risk factors and matching with similar patients, while maintaining same amount of time preparing everything. Therefore, early preparation of diagnostic testing and surgery becomes possible, and it expedites the whole diagnosis and treatment process. This ultimately may lead to better patient outcome and shorter length of stay.

The potential consequences include: saving money by targeted diagnostic tests only because the clinicians have narrowed down the patient problem list based on the patient information they have received before the patient arrives at the hospital, saving time by expediting diagnosis process, treatment planning, and logistic and personnel arrangements, and improving patient outcomes since the sooner the treatment starts, the better the patient outcome will be.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method for recommending customized emergency response for a subject, comprising:
receiving an indication that the subject requests emergency response;
determining a customized emergency response protocol for the subject, based at least on a current physiological state of the subject, wherein the protocol is different from subject to subject;
equipping an emergency response vehicle based on the customized emergency response protocol; and
dispatching the emergency response vehicle to the subject.

2. The method of claim 1, further comprising:
obtaining historical data about the subject and historical data about one or more other subjects; and
determining the customized emergency response protocol for the subject based on the current physiological state and the obtained historical data about the subject and the historical data about the one or more other subjects.

3. The method of claim 2, further comprising:
extracting risk factors for the subject and the one or more other subjects based on the current physiological state, the historical data about the subject, and the historical data about the one or more other subjects; and
determining the customized emergency response protocol for the subject based on the risk factors.

4. The method of claim 3, further comprising:
determining risk scores for the subject and the one or more other subjects based on the risk factors for the subject and the one or more other subjects; and
determining the customized emergency response protocol for the subject based on the risk scores.

5. The method of claim 4, wherein a risk score for the subject is based solely on a number of risk factors for the subject, and a risk score for at least one of the one or more other subjects is based solely on a number of risk factors for the at least one of the one or more other subjects.

6. The method of claim 4, wherein a risk score for the subject is based on a weighted summation of the risk factors for the subject, and a risk score for at least one of the one or more other subjects is based on a weighted summation of the risk factors for the at least one of the one or more other subjects.

7. The method of claim 6, wherein weights for the weighted summations are determined based on odds ratios of each risk factor.

8. The method of claim 4, wherein a risk score for the subject is based on a non-linear combination of the risk factors for the subject taking into account a correlation structure of the risk factors for the subject, and a risk score for at least one of the one or more other subjects is based on a non-linear combination of the risk factors for the at least one of the one or more other subjects taking into account a correlation structure of the risk factors for the at least one of the one or more other subjects.

9. The method of any of claims 4 to 8, further comprising:
determining a similarity measure between the risk score of the subject and risk scores of each of the one or more other subjects; and
determining the customized emergency response protocol for the subject based on the similarity measures.

10. The method of claim 9, wherein a similarity measure between the risk score of the subject and a risk score of one of the one or more other subjects is a weighted sum of a similarity measure of each of the risk factors.

11. The method of claim 10, wherein a similarity measure between a risk factor of the subject and a risk factor of one of the one or more other subjects is a reciprocal of a distance between the two risk factors.

12. The method of any of claims 9 to 11, further comprising:
ranking the similarity measures between the risk scores from one most similar to least similar or least similar to most similar; and
determining the customized emergency response protocol for the subject based on the ranked similarity measures.

13. The method of claim 12, further comprising:
retrieving diagnoses for a sub-set of the one or more other subjects corresponding a predetermined number of the highest ranked similar measures; and
determining the customized emergency response protocol for the subject based on the diagnoses of the sub-set.

14. The method of claim 13, further comprising:
computing a product of a prevalence of a diagnosis and a similarity of the subject to each of the one or more the subjects;
identifying the product with a maximum value;
identifying a candidate diagnosis of the diagnoses based on the identified maximum value; and
determining the customized emergency response protocol for the subject based on the candidate diagnosis.

15. The method of any of claims 1 to 14, wherein the protocol indicates at least one of equipment or personnel.

16. The method of any of claims 1 to 15, further comprising:
recommending at least one of a treatment, equipment, personnel, or a medical test to a healthcare facility to which the emergency response vehicle takes the subject.

17. An emergency response system (100), comprising:
a data retriever (102) including a current subject current state retriever (106) that receives an indication that a subject requests emergency response; and
an emergency response recommender (122) that determines a customized emergency response protocol for the subject, based on a current physiological state of the subject, wherein the protocol is different from subject to subject,
wherein an emergency response vehicle is equipped based on the protocol and dispatched to the subject.

18. The system of claim 17, the data retriever, further comprising:
a current subject historical data retriever (108) that obtains historical data about the subject; and
an other subject(s) historical data retriever (112) that obtains historical data about the subject,
wherein the emergency response recommender determines the customized emergency response protocol for the subject based on the current physiological state and the obtained historical data about the subject and the historical data about the one or more other subjects.

19. The system of claim 18, further comprising:
a risk factor extractor (114) that extracts risk factors for the subject and the one or more other subjects based on the current physiological state, the historical data about the subject, and the historical data about the one or more other subjects;
a risk score determiner (116) that determines risk scores for the subject and the one or more other subjects based on the risk factors for the subject and the one or more other subjects;
a similarity determiner (118) that determines a similarity measure between the risk score of the subject and the risk score of each of the one or more other subjects; and
a heath state determiner (120) that determines a candidate health state of the subject based on the similarity measures,
wherein the emergency response recommender determines the customized emergency response protocol for the subject based on the candidate health state.

20. The system of claim 19, wherein a risk score of a certain medical condition for the subject is based solely on a number of risk factors for the subject, and a risk score for at least one of the one or more other subjects for the same medical condition is based solely on a number of risk factors for the at least one of the one or more other subjects.

21. The system of claim 19, wherein a risk score for the subject is based on a weighted summation of the risk factors of a certain medical condition for the subject, and a risk score for at least one of the one or more other subjects is based on a weighted summation of the risk factors of a certain medical condition for the at least one of the one or more other subjects, wherein weights for the weighted summations are determined based on odds ratios of each risk factor.

22. The system of claim 19, wherein a risk score for the subject is based on a non-linear combination of the risk factors of a certain medical condition for the subject taking into account a correlation structure of the risk factors for the subject, and a risk score for at least one of the one or more other subjects is based on a non-linear combination of the risk factors of a certain medical condition for the at least one of the one or more other subjects taking into account a correlation structure of the risk factors for the at least one of the one or more other subjects.

23. The system of any of claims 19 to 22, wherein a similarity measure between the risk factor of the subject and a risk score of one of the one or more other subjects for the certain medical condition is a weighted sum of a similarity measure of each of the risk factors, and a similarity measure between a risk factor of the subject and a risk factor of one of the one or more other subjects is a reciprocal of a distance between the two risk factors.

24. The system of any of claims 19 to 23, wherein the health state determiner ranks the similarity measures between the risk factors from one of most similar to least similar or least similar to most similar, retrieves diagnoses for a sub-set of the one or more other subjects corresponding a predetermined number of the highest ranked similar measures, determines a product of a prevalence of the diagnoses and a similarity of the subject to the one or more the subjects, identifies the product with a maximum value, and identifies a candidate diagnosis of the diagnoses based on the identified maximum value, wherein the emergency response recommender determines the customized emergency response protocol for the subject based on the candidate health state.

25. The system of any of claims 17 to 24, wherein the protocol indicates at least one of equipment or personnel.

26. The system of any of claims 17 to 25, further comprising:
a healthcare recommender (126) that recommends at least one of a treatment, equipment, personnel, or a medical test to a healthcare facility to which the emergency response vehicle takes the subject.

27. A computer readable storage medium encoded with computer readable instructions, which, when executed by a processer, causes the processor to:
obtain a current state of a subject, historical data about the subject, and historical data about one or more other subjects;
extract risk factors for the subject and the one or more other subjects based on the current physiological state, the historical data about the subject, and the historical data about the one or more other subjects;
determine risk scores for the subject and the one or more other subjects based on the risk factors for the subject and the one or more other subjects;
determine a similarity measure between the risk factors of the subject and risk factors of each of the one or more other subjects;
rank the similarity measures between the risk factors from one of most similar to least similar or least similar to most similar;
retrieve diagnoses for a sub-set of the one or more other subjects corresponding to a predetermined number of the highest ranked similarity measures;
identify a candidate diagnosis of the diagnoses by determining a product of a prevalence of the diagnosis and a similarity of the subject to the one or more subjects and identifying the product with a maximum value, wherein the candidate diagnosis is the candidate diagnosis corresponding to the identified maximum value; and
generate a customized emergency response protocol for the subject based on the candidate diagnosis.
